(19) United States
(12) Patent Application Publication
(10) Pub. No.: US 2011/0263893 A1
(43) Pub. Date: Oct. 27, 2011

(54) PRODUCTS, PROCESS FOR PRODUCING THEM, AND THEIR USE

(75) Inventors: Jochen Ammenn, Sandhausen (DE); Sebastien Garnier, Berlin (DE)

(73) Assignee: BASF SE, Ludwigshafen (DE)

(21) Appl. No.: 13/091,566

(22) Filed: Apr. 21, 2011

Related U.S. Application Data
(60) Provisional application No. 61/326,261, filed on Apr. 21, 2010.

(51) Int. Cl. C07C 309/43

(52) U.S. Cl. 562/75

(57) ABSTRACT

Process for producing products which comprises
(A) reacting at least one compound of the general formula (I)

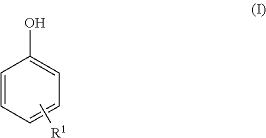

where R¹ is selected from hydrogen and C₁-C₄-alkyl,
and a sulfonating agent with one another in a molar ratio in the range from 1:0.8 to 1:1.09, based on active SO₃, and
(B) condensing the resultant intermediate with at least one aliphatic aldehyde.

The present invention relates to a process for producing products which comprises

(A) reacting at least one compound of the general formula (I)

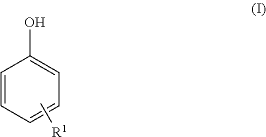

where R¹ is selected from hydrogen and C₁-C₄-alkyl,
and a sulfonating agent with one another in a molar ratio in the range from 1:0.8 to 1:1.09, based on active SO₃, and
   (B) condensing the resultant intermediate with at least one aliphatic aldehyde.

The present invention further relates to products which are obtainable by the process of the invention, and to their use for producing leather.

The production of leather requires numerous assistants, which can be used, for example, at the pretanning, tanning or retanning stages. One class of frequently used tanning or retanning agents is known as syntans. These are understood to be condensation products of sulfonated aromatics with aldehydes and other compounds, urea for example. Processes for preparing such condensation products, which are generally not pure substances but rather difficult-to-separate mixtures of numerous substances with different molecular weights, different degrees of substitution and/or different functionalities, are described in WO 2006/064054, for example.

In many cases it is observed that leathers tanned or retanned with syntans exhibit poor thermal yellowing behavior, i.e., yellow rapidly or strongly under hot conditions. The softness, too, is unsatisfactory—generally speaking, very soft leathers are not obtained, but instead only leathers with a moderate softness.

The object, then, was to provide products with which soft leathers can be made that exhibit good thermal yellowing behavior. A further object was to provide a process for producing such products. An object, finally, was to provide soft leathers having good thermal yellowing behavior.

Accordingly, the process defined at the outset has been found, also called production process of the invention for short.

The production process of the invention encompasses a plurality of steps, which are also identified below as step (A) or step (A) of the production process of the invention, step (B) or step (B) of the production process of the invention, and so on.

The production process of the invention starts from at least one compound of the general formula (I)

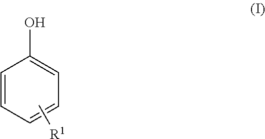

in which R¹ is selected from C₁-C₄-alkyl, as for example sec-butyl, isobutyl, isopropyl, preferably unbranched, for example methyl, ethyl, n-propyl, n-butyl, with particular preference methyl
and more particularly hydrogen.

Preferred representatives of compounds of the general formula (I) are ortho-cresol, meta-cresol, para-cresol, and, in particular, phenol.

In step (A), a compound of the general formula (I) is reacted with a sulfonating agent, such that the molar ratio of compound of the general formula (I) to active SO₃ is in the range from 1:0.8 to 1:1.09, preferably 1.00-1.09.

Examples of suitable sulfonating agents include the following: chlorosulfonic acid, sulfuric acid, disulfuric acid H₂S₂O₇, and, in particular, oleum.

In one embodiment of the present invention, step (A) is carried out at temperatures in the range from 80 to 180° C., preferably in the range from 140 to 170° C.

In one preferred embodiment of the present invention, the HCl formed during the reaction (in the case, for example, of chlorosulfonic acid as sulfonating reagent) or, in particular, water is removed during the reaction, by distillation, for example.

In one embodiment of the present invention, step (A) is carried out under atmospheric pressure. In another embodiment, step (A) may be carried out at elevated pressure, as for example in a range from 1.01 to 10 bar. In another embodiment of the present invention, step (A) is carried out under reduced pressure, as for example in a range from 20 to 100 mbar.

In one embodiment of the present invention, a solvent may be used for carrying out step (A).

In one preferred embodiment of the present invention, step (A) of the process of the invention is carried out without solvent. In such embodiments, in the cases where compound of the general formula (I) is solid at room temperature, operation is such that compound of the general formula (I) is used in melted form.

In one embodiment of the present invention the procedure is such that compound of the general formula (I) is introduced in liquid form and sulfonating reagent is added.

In another embodiment of the present invention, the procedure adopted may be such that sulfonating reagent is introduced and compound of the general formula (I) is added in preferably liquid form.

The reaction in step (A) may be carried out over a time in the range from 10 minutes to 24 hours, with 4 to 10 hours being preferred.

The reaction in step (A) of the production process of the invention is carried out preferably under inert gas, as for example under nitrogen. The reaction in step (A), however, may also be carried out in the presence of air.

The intermediate obtained in step (A) is generally a mixture in which compound of the general formula (II)

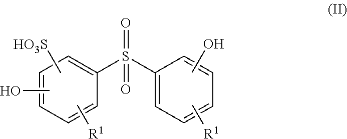

is a principal constituent, i.e., the most important constituent by weight.

The compound in question is preferably

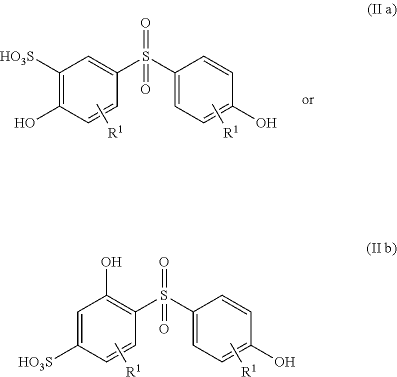

or mixtures of the compounds identified above.

In one embodiment of the present invention, intermediate obtained according to step (A) may be contaminated with compounds of the general formulae (III a), (III b), (IV) and/or (V).

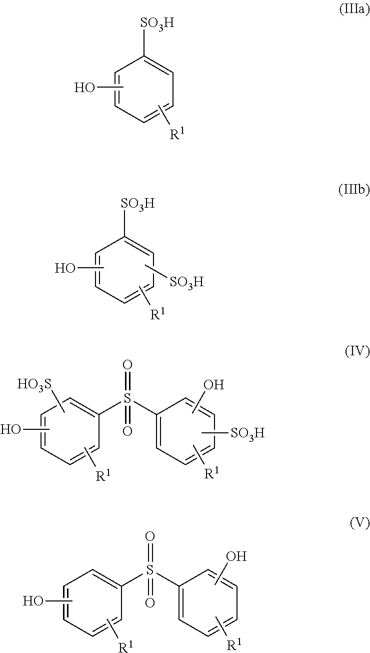

The fraction of contamination by compounds of the general formula (III a), (III b), (IV), and (V) is in the majority of cases not more than 60% by weight, based on intermediate as a whole.

For carrying out the production process of the invention, step (B) is additionally carried out. In step (B), the intermediate obtained from step (A) is condensed with at least one aliphatic aldehyde.

Examples of suitable aliphatic aldehydes include the following: propionaldehyde, acetaldehyde, and, in particular, formaldehyde. Mixtures of two different aliphatic aldehydes, such as of acetaldehyde and formaldehyde, for example, are likewise suitable.

Aliphatic aldehyde, especially acetaldehyde, may be used, for example, in aqueous solution or as a trimer.

Formaldehyde may be used, for example as a gas, as polymeric formaldehyde or in aqueous solution, as formalin, for example.

In one embodiment of the present invention, intermediate from step (A) is reacted with aliphatic aldehyde in a molar ratio in the range from 1:0.5 to 1:4, preferably 1:0.6 to 1:3.5. In calculating the number of moles of intermediate from step (A) here it is assumed that the reaction to form compound of the general formula (II) would be as complete as possible and that no compounds of the general formula (IIIa) to (V) are present.

In one embodiment of the present invention, step (B) is carried out at temperatures in the range from 50 to 140° C., preferably 60 to 120° C.

In one embodiment of the present invention, step (B) is carried out under atmospheric pressure. In another embodiment, step (B) may be carried out at elevated pressure, as for example in a range from 1.01 to 10 bar.

In one embodiment of the present invention, step (B) may be carried out without solvent. Preferably, however, step (B) is carried out in aqueous solution.

The reaction in step (B) may be carried out over a time in the range from 10 minutes to 24 hours, with 20 minutes to 6 hours being preferred.

In one embodiment of the present invention, it is possible in step (B) to add at least one further, preferably aromatic, compound which participates in the condensation reaction, an example being 2,4′-dihydroxydiphenyl sulfone or 4,4′-dihydroxydiphenyl sulfone.

It is preferred not to add any other organic compound in step (B). It is particularly preferred to add neither an aromatic compound nor urea in step (B).

In one embodiment of the present invention, partial or complete neutralization may take place after step (A) has been carried out and before or after condensation in step (B) has taken place.

For neutralizing it is possible, for example, to use organic amines, ammonia or, preferably, basic alkali metal compounds. Examples of organic amines are ethylamine, diethylamine, triethylamine, n-propylamine, di-n-propylamine, N,N-diethanolamine, ethanolamine, N-methylethanolamine, N,N-dimethylethanolamine, N-methyl-N,N-diethanolamine, N-(n-propyl)ethanolamine, N-(n-butyl)ethanolamine and N-(n-butyl)-N,N-diethanolamine.

Examples of basic alkali metal compounds are carbonates, hydrogencarbonates, and, in particular, hydroxides of alkali metals, more particularly of potassium or sodium. Potassium hydroxide and sodium hydroxide are particularly preferred. Basic alkali metal compound can be used preferably in aqueous solution.

In one embodiment of the present invention, the partial or complete neutralization is carried out at temperatures in the range from zero to 90° C., preferably 20 to 30° C.

In one embodiment of the present invention, the intermediate from step (A) can be purified. It is preferred, however, not to carry out purification and to use the intermediate from step (A) either immediately in step (B) or to use it in step (B) following partial or complete neutralization.

A product of the invention is obtained which can be used, for example, for producing leather. By product of the invention is meant the organic fractions, in other words free acids or else salts. Product of the invention can be employed in aqueous formulation or first isolated, as for example by evaporative concentration or, more particularly, by spray drying of the reaction mixtures obtainable according to step (B).

In one embodiment of the present invention, product of the invention may have an average molecular weight M_{w} in the range from 1000 to 10 000 daltons, preferably 2000 to 5000 daltons. The average molecular weight may be determined, for example, by gel permeation chromatography (GPC), taking a syntan, for example, as a standard. An example of a suitable solvent is water.

The present invention further provides for the use of product of the invention for producing leather. The present invention further provides a process for producing leather using product of the invention. The present invention further provides leather produced using product of the invention.

In one embodiment of the present invention the process of the invention is practiced for the production of leather as a tanning process, also referred to below as tanning process of the invention, and preferably as a retanning process, also referred to below as retanning process of the invention.

The tanning process of the invention is generally practiced such that product of the invention is added in one portion or in two or more portions immediately prior to or else during the tanning step.

The tanning process of the invention is carried out preferably at a pH from 2.5 to 11, preferably to 5, and it is frequently observed that the pH rises by around 0.3 to three units during the implementation of the tanning process of the invention.

The tanning process of the invention is generally carried out at temperatures of 10 to 45° C., preferably at 20 to 30° C. A duration of 10 minutes to 12 hours has been found appropriate, with one to three hours being preferred. The tanning process of the invention can be carried out in any desired customary tannery vessels, as for example by agitation in barrels or in rotated drums.

In one embodiment of the present invention, a total of 0.01% to 10% by weight of product of the invention is used, based on the solids content of product of the invention on the one hand and on the shaved weight on the other hand, with 0.5% to 5% by weight being preferred.

In one version of the tanning process of the invention, aqueous formulation of the invention is used together with one or more conventional tanning materials, as for example with chrome tannings, mineral tannings, syntans, polymeric tannings or vegetable tannings, as described for example in Ullmann's Encyclopedia of Industrial Chemistry, volume A15, pages 259 to 282 and particularly page 268 ff., 5th edition, (1990), Verlag Chemie Weinheim. The product of the invention: conventional tanning or sum of conventional tannings weight ratio is advantageously from 0.01:1 to 100:1 (based in each case on the solids contents).

In one variant of the tanning process of the invention, product of the invention is used together with one or more fatliquors or oleophilic components.

In one version of the tanning process of the invention, product of the invention is added in one portion or in two or more portions before or during pretannage. An addition at the pickling stage is also conceivable.

The retanning process of the invention is carried out starting from semifinished products tanned conventionally, i.e., for example, with chrome tannings, mineral tannings, polymer tannings, aldehydes, syntans or resin tannings, or produced in accordance with the invention as described above. To carry out the retanning process of the invention, at least one product of the invention is caused to act on semifinished products, i.e., such product is treated with at least one product of the invention.

The retanning process of the invention can be carried out under otherwise customary conditions. It is advantageous to select one or more, i.e., 2 to 6, treatment steps, and rinsing with water may be carried out between the treatment steps. The temperature in the individual treatment steps is in each case from 5 to 60° C., preferably 20 to 45° C. It is advantageous to use one or more further agents that are typically used during retanning, examples being fatliquors, polymeric tannings, and fatliquoring agents based on acrylate and/or methacrylate, retannings based on vegetable tannings, fillers, leather dyes or emulsifiers.

In one embodiment of the present invention, the retanning process of the invention is carried out at a pH in the range from 4 to 9.

For the retanning process of the invention, a duration of 10 minutes to 12 hours has been found appropriate, with one to three hours being preferred. The retanning process of the invention can be carried out in any desired customary tannery vessels, as for example by agitation in barrels or in rotated drums.

In one embodiment of the retanning process of the invention, a total of 0.01% to 10% by weight of product of the invention is used, based on the shaved weight on the other hand, with 0.5% to 5% by weight being preferred.

Additionally provided by the present invention is leather produced by the process of the invention. Leather of the invention is notable for high fullness, softness, and good thermal yellowing properties, i.e., a low tendency toward thermal yellowing, and by other good service properties. Leather of the invention is suitable, for example, for producing footwear upper leather, apparel items such as jackets, coats or pants, and also of furniture.

The invention is illustrated by working examples.

### I. Production of an Inventive Product (IP.1)

### I.1 Sulfonation According to Step (A.1)

In a 4 l four-necked flask, 409.34 g of phenol (4.35 mol) were melted at 45° C. With stirring, 404.4 g of 20% strength oleum (corresponding to 4.35 mol of SO₃) were added over the course of 15 minutes. The temperature rose to up to 134° C. A subatmospheric pressure of 35 mbar was applied and the mixture was heated to 160° C. and stirred at 160° C. for a time of five hours, during which the water formed was removed by distillation (78.2 g, 4.34 mol).

The batch was then cooled to 50° C., and 500 g or water were added over a period of 25 minutes. This gave an aqueous solution of an intermediate which was composed primarily of

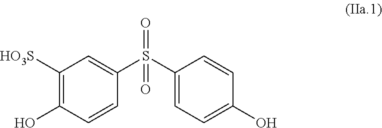

### I.2 Condensation According to Step (B.1)

300 g of a solution from I.1 were introduced, corresponding to about 0.57 mol of compound of the formula (II a.1) or isomers thereof, and this solution was cooled to 0° C. Over the course of 13 minutes, with cooling, 95.7 g of 50% strength by weight aqueous sodium hydroxide solution were added, the temperature remaining at 0° C. The pH was 8.1. Over the course of 8 minutes, 36.3 g of a 30% strength aqueous formaldehyde solution were added, corresponding to 0.36 mol of formaldehyde. During the addition, the temperature rose to 18° C. After the end of the addition of the formaldehyde solution, the batch was heated to 100° C. over the course of 15 minutes and was stirred at 100° C. for 5 hours. Subsequently it was cooled to room temperature and a pH of 5.1 was set with formic acid. This gave an aqueous solution of product of the invention (IP.1).

### I.3 Condensation According to Step (B.2)

300 g of a solution from I.1 were introduced, corresponding to about 0.57 mol of compound of the formula (II a.1) or isomers thereof, and this solution was cooled to 0° C. Over the course of 13 minutes, with cooling, 95.7 g of 50% strength by weight aqueous sodium hydroxide solution were added, the temperature remaining at 0° C. The pH was 8.1. Over the course of 18 minutes, 181 g of a 30% strength by weight aqueous formaldehyde solution were added, corresponding to 1.8 mol of formaldehyde. During the addition, the temperature rose to 18° C. After the end of the addition of the formaldehyde solution, the batch was heated to 100° C. over the course of 15 minutes and was stirred at 100° C. for 5 hours. Subsequently it was cooled to room temperature and a pH of 5.1 was set with formic acid. This gave an aqueous solution of product of the invention (IP.2).

### II. Use of Products of the Invention

Production of footwear upper leather (general instructions)

Figures in % are always % by weight and are based on the shaved weight. The figures in % in the case of aqueous formulations relate always to the solids fraction or active compound fraction, respectively, unless expressly indicated otherwise.

As a tanning agent for comparison a syntan was used.

A commercially available bovine wet blue (from Packer, USA) was shaved to a thickness of 1.5 to 1.8 mm. The butt region was cut into three strips of around 1700 g each. The strips were then admixed in a drum (50 l) and a float length of 200% (based on shaved weight) with 1.5% by weight of sodium formate and 0.5% of sodium bicarbonate and also 1% of a naphthalenesulfonic acid-formaldehyde condensation product prepared according to U.S. Pat. No. 5,186,846, in the “Dispersant 1” example, at 10-minute intervals. After 70 minutes, the float was dropped. The strips were then allocated to separate milling drums 1 to 3.

Together with 100% of water, the milling drums 1 to 3 were admixed at 25 to 35° C. with, in each case, 8% of product IP.1 or IP.2 of the invention, or comparative tanning agent V-GM.3 according to Table 1. Following a milling time of 20 minutes, 0.7% of formic acid was added.

For light fastness and thermal yellowing, two strips were cut off and dried. The light fastness was assessed after 24 h exposure to light; the thermal yellowing after 72 h at 100° C.

The rest of the leather was admixed after 60 minutes with in each case 2% by weight of 50% by weight (solids content) aqueous solution of dyes in which the solids had the following composition:

70 parts by weight of dye from EP-B 0 970 148, Example 2.18,
30 parts by weight of Acid Brown 75 (iron complex), Colour Index 1.7.16

This was followed by a further 30 minutes of drumming.

Subsequently, in a plurality of steps, the leathers were acidified down to a pH of 3.6 to 3.8 using formic acid at 0.3% to 0.5%. After 20 minutes, the float was assessed for exhaustion and dropped. The leathers were washed with 200% of water.

Thereafter, in 100% of water (50° C.), 4% of a fatliquor according to WO 03/023069, Example A was added. After 20 minutes, the leathers were acidified down to a pH of 3.8 with 1% formic acid.

The leathers obtainable in this way were washed, dried, staked, treated for 30 seconds in a vacuum dryer at 60° C., and assessed according to the test criteria set out in Table 1. The leathers L.1 and L.2 of the invention, and the comparative leather V-L.3, were obtained. Evaluation took place in accordance with a rating system from 1 (very good) to 5 (deficient).

TABLE 1

| Leather | L.1 | L.2 | V-L.3 |
|---|---|---|---|
| Product used (8%) | IP.1 | IP.2 | V-GM.3 |
| Heat resistance | 2 | 3 | 4 |
| Light fastness | 3 | 2 | 3 |
| Depth of shade | 3 | 3 | 3 |
| Fullness | 2 | 3 | 4 |
| Softness | 2 | 1 | 3 |

1.-6. (canceled)

7. A process for producing products which comprises
(A) reacting at least one compound of the general formula (I) 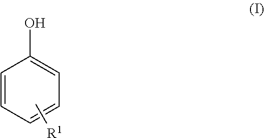
where R¹ is selected from hydrogen and C₁-C₄-alkyl,
and a sulfonating agent with one another in a molar ratio in the range from 1:0.8 to 1:1.09, based on active SO₃, and
(B) condensing the resulting intermediate with at least one aliphatic aldehyde.

8. The process according to claim 7, wherein formaldehyde is selected as aliphatic aldehyde.

9. The process according to claim 7, wherein partial or complete neutralization is carried out after step (A) has been carried out and before or after condensation has been carried out in step (B).

10. The process according to claim 8, wherein partial or complete neutralization is carried out after step (A) has been carried out and before or after condensation has been carried out in step (B).

11. The process according to claim 7, wherein no additional aromatic compounds are used for the condensation in step (B).

12. The process according to claim 10, wherein no additional aromatic compounds are used for the condensation in step (B).

13. A product obtainable by the process according to claim 7.

14. A product obtained by the process according to claim 12.

15. A process for producing leather which comprises utilizing the product as claimed in claim 13.
